(19) United States
(12) Patent Application Publication
(10) Pub. No.: US 2026/0096716 A1
(43) Pub. Date: Apr. 9, 2026

(54) MEDICAL DEVICE AND METHODS

(71) Applicant: Boston Scientific Scimed, Inc., Maple Grove, MN (US)

(72) Inventors: Andrew James Schaubhut, Bolton, MA (US); Scott E. Brechbiel, Johnston, RI (US); Julie Gagnon, Providence, RI (US)

(73) Assignee: Boston Scientific Scimed, Inc., Maple Grove, MN (US)

(21) Appl. No.: 19/349,225

(22) Filed: Oct. 3, 2025

Related U.S. Application Data
(60) Provisional application No. 63/704,322, filed on Oct. 7, 2024.

(51) Int. Cl. A61B 1/00; A61B 1/005; A61B 1/12; H01R 13/73

(52) U.S. Cl. CPC A61B 1/00124; A61B 1/00066; A61B 1/0011; A61B 1/00128; A61B 1/0057; A61B 1/12; H01R 13/73; H01R 2201/12

(57) ABSTRACT

Devices, systems, and methods for a medical device are disclosed. The medical device may include a handle configured to connect with an electrical source. The handle may include a hub, a wire, and an actuator. The hub may include a fluid port, an electrical connector, and an electrically conductive tube coupled with the electrical connector. The wire may be electrically coupled to the electrically conductive tube. The actuator may be coupled with the hub and the wire. The actuator may be configured to adjust relative to the hub and in response, adjust the wire relative to the electrically conductive tube.

### CROSS-REFERENCE TO RELATED APPLICATIONS

This application claims the benefit of U.S. Provisional Patent Application Serial No. 63/704,322 filed on October 7, 2024, the disclosure of which is incorporated herein by reference.

### TECHNICAL FIELD

The present disclosure pertains generally, but not by way of limitation, to medical devices and systems, and methods of assembly and treatment. More particularly, the present disclosure relates to medical devices used, for example, in endoscopic procedures.

### BACKGROUND

Various devices, systems, and methods have been developed to access body lumens and treat issues within or at body lumens, including, but not limited to, body lumens of the biliary tract of a subject (e.g., a patient). These devices and systems are manufactured by any one of a variety of different manufacturing methods and may be used according to any one of a variety of methods for a variety of applications. Of the known medical devices, delivery systems, and methods, each has certain advantages and disadvantages. There is an ongoing need to provide alternative medical devices and delivery devices as well as alternative methods for manufacturing and using medical devices and delivery devices, such as those susceptible to migration in the anatomy.

### BRIEF SUMMARY

This disclosure provides design, material, manufacturing method, and use alternatives for medical devices.

In a first example, a handle for a medical device configured to connect with an electrical source may include a hub comprising a fluid port, an electrical connector, and an electrically conductive tube coupled with the electrical connector. The handle may further include a wire electrically coupled to the electrically conductive tube and an actuator coupled with the hub and the wire, the actuator may be configured to adjust relative to the hub and in response, adjust the wire relative to the electrically conductive tube.

Additionally or alternatively to any of the examples above, the wire may comprise a bend configured to engage an inner surface of the electrically conductive tube.

Additionally or alternatively to any of the examples above, the handle may further include a crimp connector coupled with the wire, wherein the crimp connector is configured to engage an inner surface of the electrically conductive tube.

Additionally or alternatively to any of the examples above, the electrical connector may be located distal of the fluid port and a lumen in communication with the fluid port may be electrically isolated from the electrical connector.

Additionally or alternatively to any of the examples above, the electrical connector may comprise threads configured to engage the hub and a distal end of the electrical connector electrically coupled with the electrically conductive tube.

Additionally or alternatively to any of the examples above, the wire may be configured to longitudinally and rotationally adjust relative to the electrically conductive tube.

Additionally or alternatively to any of the examples above, the electrical connector may comprise a slot extending through a distal end of electrical connector, the hub may comprise a through-hole configured to receive the electrical connector, the slot may be configured to receive the electrically conductive tube, and a fastener may be configured to be positioned within the through-hole and engage threads extending around the electrical connector to maintain electrical contact between the electrical connector and the electrically conductive tube received within the slot.

Additionally or alternatively to any of the examples above, the electrical connector may comprise a slot extending through a distal end of electrical connector, the hub may comprise a hole, a portion of the electrical connector along the slot may define two legs each having a barbed portion, and the two legs may be configured to create a press fit engagement with an inner surface of the hole and secure the electrically conductive tube at the barbed portion between the electrical connector and the inner surface of the hole.

Additionally or alternatively to any of the examples above, the electrical connector may comprise a recess configured to receive the electrically conductive tube and a portion of a housing of the hub.

Additionally or alternatively to any of the examples above, the electrical connector may comprise a recess configured to receive the electrically conductive tube, the recess is perpendicular to a longitudinal axis of the electrical connector, the hub may comprise an electrical connector port configured to secure the electrical connector at a non-perpendicular orientation relative to a longitudinal axis of a lumen in the hub configured to receive the wire, and the electrical connector may be configured to receive the electrically conductive tube in the recess and electrically engage an outer surface of the electrically conductive tube.

Additionally or alternatively to any of the examples above, the electrical connector may include a crimp electrically coupling the electrical connector with the electrically conductive tube.

Additionally or alternatively to any of the examples above, the electrical connector may comprise an opening configured to receive the electrically conductive tube and electrically couple the electrical connector with the electrically conductive tube via a press-fit of the electrical connector into the opening.

Additionally or alternatively to any of the examples above, the electrical connector may be configured to receive the electrically conductive tube, a lumen in fluid communication with the fluid port, and an insulating tube positioned around the lumen and configured to urge the electrically conductive tube into electrical contact with the electrical connector.

In another example, a medical device may comprise a hub having a proximal end and a distal end, the hub comprising a fluid port and an electrical connector. The medical device may further include an elongate shaft extending in a distal direction from the hub, an end effector in electrical communication with the electrical connector and configured to be positioned proximate a distal end of the elongate shaft, and an actuator coupled with the proximal end of the hub, the actuator is configured to adjust relative to the hub and in response, adjust the end effector, wherein the actuator and the end effector may be configured to longitudinally and rotationally adjust relative to the hub.

Additionally or alternatively to any of the examples above, the medical device may further include a wire electrically connected with the electrical connector and mechanically connected to the actuator, wherein actuation of the actuator relative to the hub may cause the wire and the end effector to adjust.

Additionally or alternatively to any of the examples above, the hub may further comprise an electrically conductive tube electrically coupled with the electrical connector and the wire may be electrically coupled with the electrically conductive tube and the actuation of the actuator adjusts the wire relative to the electrically conductive tube.

Additionally or alternatively to any of the examples above, the wire may form at least a portion of the end effector and may be configured to cut tissue of a patient.

Additionally or alternatively to any of the examples above, the hub may further comprises an electrically conductive tube electrically coupled with the electrical connector and a fluid lumen in communication with the fluid port and electrically insulated from the electrically conductive tube.

In another example, a method of assembling a medical device may include securing an electrically conductive tube in a hub having a fluid port, electrically coupling an electrical connector with the electrically conductive tube, and coupling an actuator with the hub, wherein the actuator coupled with the hub may be configured to adjust relative to the hub.

Additionally or alternatively to any of the examples above, securing the electrically conductive tube in the hub may comprise one or more of over-molding the electrically conductive tube in the hub, press-fitting the electrically conductive tube in the hub, coupling two or more components of the hub around the electrically conductive tube, and engaging the electrically conductive tube between the electrical connector and a fastener coupled with the electrical connector.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative medical device extending into a body of a subject;

FIG. 2 is a schematic enlarged view of the portion of the illustrative medical device extending into the body of the subject within circle-2 in FIG. 1;

FIG. 3 is a schematic diagram of an illustrative medical system;

FIG. 4 is a schematic side view of a portion of an illustrative configuration of a medical device;

FIG. 5 is a schematic cross-section view of the portion of the medical device depicted in FIG. 4;

FIG. 6 is a schematic partial cross-section perspective view of an illustrative configuration of a portion of a medical device;

FIG. 7 is a schematic cross-section view of an illustrative configuration of a medical device;

FIG. 8 is a schematic partial cross-section view of an illustrative configuration of a medical device;

FIG. 9A is a schematic partial cross-section view of a portion of an illustrative configuration of a medical device;

FIG. 9B is a schematic partial cross-section view of the portion of the medical device depicted in FIG. 9A, taken along line 9B-9B;

FIG. 10 is a schematic cross-section view of an illustrative configuration of a medical device;

FIG. 11A is a schematic partial cross-section view of a portion of an illustrative configuration of a medical device;

FIG. 11B is a schematic cross-section view of the portion of the medical device depicted in FIG. 11A, taken along line FIGS. 11B-11B;

FIG. 12 is a schematic cross-section view of a portion of an illustrative configuration of a medical device;

FIG. 13 is a schematic side view of an illustrative configuration of an electrical connector;

FIG. 14 is a schematic cross-section view of an illustrative configuration of a medical device;

FIG. 15 is a schematic cross-section of a portion of an illustrative configuration of a medical device;

FIG. 16 is a schematic partial cross-section view of a portion of an illustrative configuration of a medical device; and

FIG. 17 is a schematic box diagram of an illustrative method of assembling a medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Endoscopic retrograde cholangiopancreatography (ERCP) may be used to diagnose and treat conditions of the common bile duct, including, for example, gallstones, inflammatory strictures, leaks (e.g., from trauma, surgery, etc.), and cancer. In ERCP procedures, a physician may view, through an endoscope, the inside of the stomach and/or the duodenum. Often, dyes may be injected (e.g., via a lumen of a sphincterotome or other device) into the ducts in the biliary tree and pancreas so that the area can be seen using X-rays. These procedures may necessitate gaining and keeping access to the papilla of Vater, the common bile duct, and/or the pancreatic duct, which may be technically challenging, may require extensive training and practice to gain proficiency, and may require one or more expensive tools in order to perform.

During an ERCP procedure, a number of steps are typically performed while the patient is often sedated and/or anaesthetized. For example, an endoscope or duodenoscope may be inserted through the mouth, down the esophagus, into the stomach, through the pylorus into the duodenum, to a position at or near the papilla of Vater (also referred to as the ampulla of Vater), which is the opening of the common bile duct and the pancreatic duct. Such orientation may allow a clinician to obtain an image of the medial wall of the duodenum, where the papilla of Vater is located, even though the distal tip of the viewing device is beyond the opening. Once the papilla or a target area in the duodenum is visually located, an accessory medical device, such as sphincterotome or other accessory medical device, may be extended out a side opening or window of the endoscope or duodenoscope for facilitating access through the papilla and into a desired one of the common bile duct and the pancreatic duct. Once access to the common bile duct and/or the pancreatic duct is attained, an accessory medical device may be inserted into the common bile duct and/or the pancreatic duct to perform a treatment.

Although the configurations of the accessory medical device discussed herein may be discussed relative to an accessory medical device configured as a sphincterotome, the concepts disclosed herein may be used with respect to other accessory medical devices configured to couple with a power source. Example accessory medical devices configured to couple with a power source (e.g., to provide power to an end effector) include, but are not limited to, sphincterotomes, snares, baskets, graspers, forceps, and/or other suitable accessory medical devices.

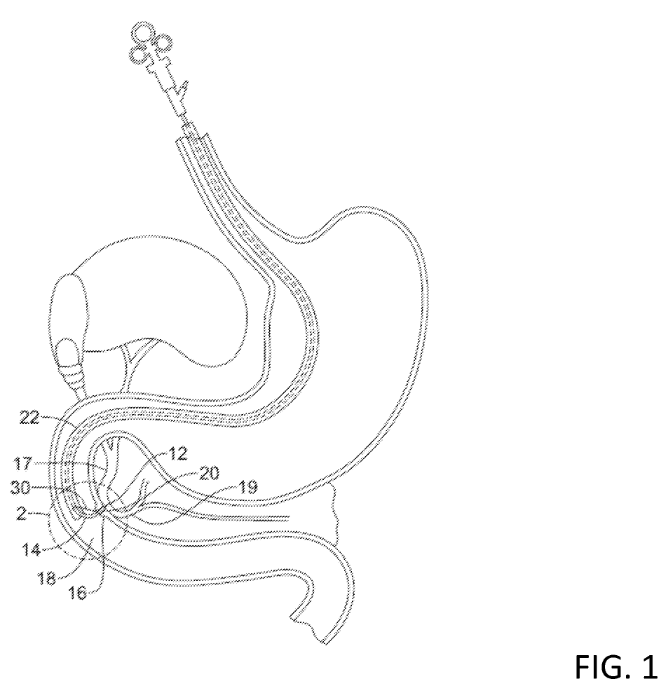

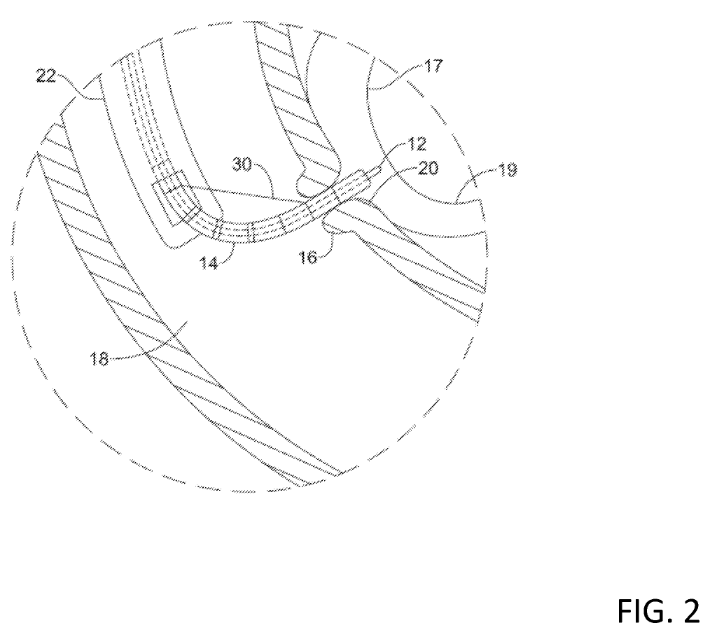

Turning to the Figures, FIGS. 1 and 2 depict a selective cannulation during an ERCP procedure, which includes a guidewire 12 and/or an accessory medical device 14 (e.g., an endoscopic accessory device, such as a sphincterotome, and/or other suitable accessory medical device) being passed towards, against, and/or through a body lumen such as the major papilla 16 (e.g., ampullary entry) near the descending duodenum 18 to access the Sphincter of Oddi Complex 20. During the cannulation procedure, a distal portion of a medical device 22 (e.g., an endoscope, duodenoscope, guide catheter, etc.) may be positioned within the descending duodenum 18. The guidewire 12 and the accessory medical device 14 may be advanced through a working channel (e.g., a lumen) of the medical device 22 towards the major papilla 16. Additionally, the guidewire 12 and/or the accessory medical device 14 may be advanced against and/or through the major papilla 16 to one of the common bile duct 17 and the pancreatic duct 19.

Accessing the papilla 16, the common bile duct 17, and/or the pancreatic duct 19 may be difficult because the openings are small compared to a size (e.g., a diameter or other size measurement) of many accessory medical devices, the openings may be closely located, the openings may be completely collapsed/closed, the openings may extend into the descending duodenum 18 at an angle that may be difficult to visualize and/or access, and/or it may be difficult to control a position of a distal tip of the accessory medical device 14. Thus, a medical professional may be required to manipulate the accessory medical device 14 and the guidewire 12 by manually rotating the medical device 22, pulling on a wire 30 (e.g., a wire filament, etc.) to adjust the accessory medical device 14 in a single direction, and/or use an elevator and/or other suitable ramped surface within the distal end of the medical device 22 in an attempt to better align or orient the accessory medical device 14 and/or the guidewire 12 with respect to the medical device 22 and the openings of the papilla 16, the common bile duct 17, and/or the pancreatic duct 19.

ERCP procedures and/or other procedures using an endoscopes, duodenoscope, colonoscopes, and/or other scopes may use the services of a medical practitioner to operate the scope along with at least one other medical professional. In one example, a medical practitioner may be focused on the operation of the scope to maintain a position thereof within a subject (e.g., a patient) while a nurse, medical technician, medical practitioner, and/or other medical professional performs an action with the accessory medical device (e.g., with an end effector of the accessory medical device). Verbal communication between the medical practitioner operating the scope and others involved in the procedure may be required to achieve a desired result from the procedure. Often this communication can be tedious and may not result in the exact actions or results desired and/or may increase time to perform the procedure. Alternatively, a medical professional may operate the scope and the accessory medical device. For example, the medical professional may control an operation of a scope with his/her left hand (or right hand) and may control operation of the accessory medical device used in conjunction with the scope with his/her right hand (or left hand), but the configuration of current handles of accessory medical devices used with scopes can make it difficult to control operation of the accessory medical devices with a single hand.

Handles of accessory medical devices used with scopes may have a first portion coupled with an elongate wire and a second portion coupled with an elongate shaft or tube through which the wire extends. The first portion or component thereof may be longitudinally and/or rotationally adjusted relative to the second portion. Further, the first portion may include an electrical connector configured to be conductively coupled with the wire and a power source to apply power to the wire. However, because the electrical connector is located at the first portion of the accessory medical device that longitudinally and/or rotationally adjusts relative to the second portion, the electrical connector and a connection to the power source may have to longitudinally and/or rotationally adjust with the first port, which may complicate a user operating the accessory medical device with a single hand. As such, an accessory medical device and components thereof that include a handle with an adjustable portion configured to adjust relative to a further portion of the handle without requiring components connected to the handle to adjust with the adjustable portion are desirable.

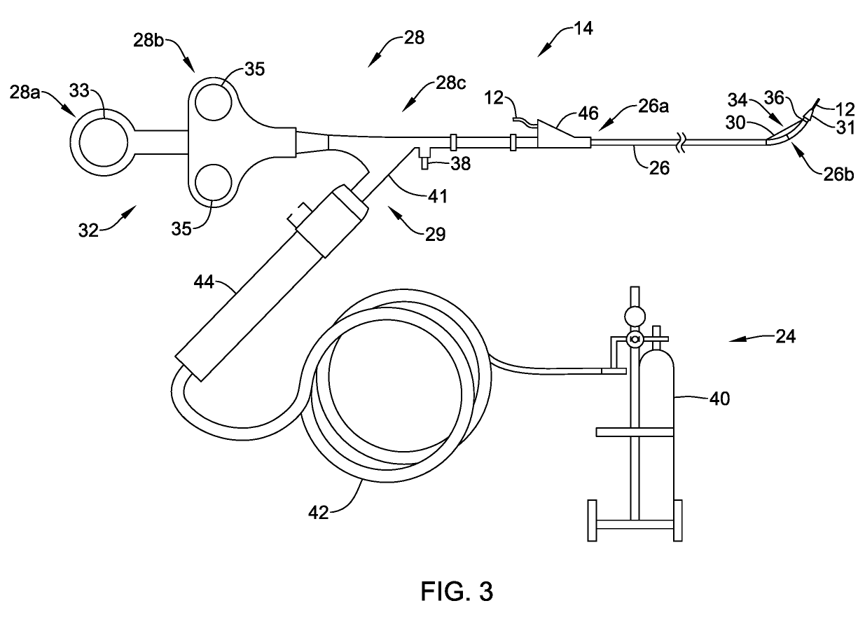

FIG. 3 schematically depicts an illustrative system 24 for accessing a body lumen. In some cases, the system 24 may include, among other components, the accessory medical device 14 with a flexible elongate tube 26. The tube 26 may have a proximal end 26a and a distal end 26b. The distal end 26b of the tube 26 may have a distal tip 31 extending distal of a distal end 36 of the wire 30 and may be configured to be directed toward and/or through an opening of a body lumen. The proximal end 26a of the tube 26 may be connected to, coupled with, or otherwise in communication with a handle 28. Although not depicted in FIG. 3, the system 24 may include or be used with a medical device, such as a scope (e.g., the medical device 22 and/or other suitable scope).

The handle 28 may be any suitable type of handle. As schematically depicted in FIG. 3, the handle 28 may have a thumb hole 33 and two finger holes 35 configured to receive fingers from one hand of the user. A first portion 28a of the handle 28 may include the thumb hole 33, a second portion 28b of the handle 28 may include the finger holes 35, and a third potion 28c of the handle 28 may be configured to releasably coupled with one or more procedural components. In some examples, the third portion 28c of the handle may a fluid port 41, an electrical connector 38, and/or other features configured to releasably coupled with procedural components. In some examples, the first portion 28a and/or the second portion 28b of the handle 28b may be devoid of features configured to couple with procedural components. In operation, the first portion 28a of the handle 28 may be manipulated longitudinally relative to the second portion 28b and the first and second portions 28a, 28b of the handle 28 may be manipulated (e.g., rotationally) relative to the third portion 28c of the handle 28 to control movement of the wire 30. In some examples, the first and second portions 28a, 28b of the handle 28 may be considered an actuator 32 and the third portion 28c of the handle 28 may be considered a hub 29, where the actuator 32 is coupled with the hub 29 and configured to adjust relative to the hub 29 to adjust the wire 30 relative to the hub 29 and/or the tube 26. Although the second portion 28b and the third portion 28c are depicted as being proximate one another, it is contemplated that second portion 28b and the third portion 28c of the handle may be spaced from one another by a tube, shaft, and/or other suitable component of the accessory medical device 14. Further, the hub 29 may be part of the handle 28 (e.g., as the third portion 28c) or separate from the handle 28. Although not required, the handle 28, as illustrated, may be held and/or operated with a single hand of a user. Other suitable configurations of the first portion 28a, the second portion 28b, and the third portion 28c of the handle 28 are contemplated.

The wire 30 may extend along the elongate tube 26 and may be energized (e.g., by a power source and/or procedure component coupled with the handle 28) for the purpose of cutting tissue within a subject. The wire 30 may have a proximal end (not shown) connected to the first portion 28a of the handle 28, and a proximal portion extending at least partially within the tube 26. The wire 30 also may have or be in communication with an end effector portion or distal portion 34 (e.g., a cutting portion configured to cut tissue of a patient, as depicted in FIG. 3, a snare, a grasper, a forceps, etc.) extending external to the elongate tube 26, and a distal end 36 of the wire 30 may be connected to the distal end 26b of the elongate tube 26.

The wire 30 may be received within a lumen of the tube 26 at least along a proximal portion of the wire 30 and may be configured to slide within the lumen. In some cases, the first portion 28a of the handle 28 may be coupled to the wire 30 and manipulated to slide the wire 30 along the tube 26 (e.g., within the lumen of the tube 26). In one example, the first portion 28a of the handle 28 may be longitudinally adjusted relative to the second portion 28b and/or the third portion 28c of the handle 28 to apply and remove tension to and/or from the wire 30.

Sliding or adjusting the wire 30 may result in the distal end 26b of the elongate tube 26 moving responsively to the sliding of the wire 30. As such, the first portion 28a and/or the second portion 28b of the handle 28 may be manipulated to control or adjust a position of the distal end 26b of the elongate tube 26, for example, to direct the distal end 26b toward the opening of a body lumen. In some cases, natural tension in or acting on the tube 26 and/or the wire 30 may result in the wire 30 returning to a relaxed position or state once a tension applied to the wire 30 via the handle 28 is released.

The wire 30 may be utilized to electrically cut or remove tissue of the subject (e.g., to cut and cauterize tissue) and may be considered a cautery wire. As such, the handle 28 or other suitable portion of the accessory medical device 14 may include the electrical connector 38 for an energy or power source (e.g., a radiofrequency energy source, or the like) to energize the wire 30 and facilitate cutting tissue. As depicted in FIG. 3 and discussed, the electrical connector 38 may be located at the third portion 28c of the handle 28 such that the wire 30 is configured to adjust longitudinally and/or rotationally relative to the electrical connector 38. When the first and/or second portions 28a, 28b of the handle 28 are manipulated to adjust a position of the distal end 26b of the tube 26 and/or utilize the wire 30 for cutting tissue, the wire 30 may be spaced from the tube 26 and electrified to facilitate cutting tissue at or adjacent to the opening of the body lumen.

The distal tip 31 of the tube 26 may be flexible and configured to adjust in response to steering with or adjustment of the first portion 28a and/or the second portion 28b of the handle 28. The distal end 26b of the elongate tube 26 of the accessory medical device 14 may be configured to bend or otherwise adjust in response to actuation of the wire 30 or in response to being advanced along another device (e.g., an elevator of a duodenoscope and/or other suitable device) that directs a position of the elongate tube 26. Additionally or alternatively, the distal tip 31 of the tube 26 may be a steerable via one or more steering wires or other suitable steering components that may be steered to facilitate precisely controlling a distal end of the accessory medical device 14 while the accessory medical device 14 is within a subject.

In addition to a lumen for receiving the wire 30, the accessory medical device 14 may include one or more other lumens extending from at least the proximal end 26a of the tube 26 to the distal end 26b of the tube 26. In some cases, one or more of the lumens may have a distal opening through the distal tip 31 of the elongate tube 26.

In some examples, a lumen of the one or more lumens of the elongate tube 26 may have a fluid connection at a proximal end for a fluid source (e.g., a pressurized fluid source 40 such as a syringe for providing contrast fluid, etc.) The lumen may be configured to apply a fluid from the fluid source 40 through an opening at the distal end 26b of the elongate tube 26 to a body lumen of the subject.

Although FIG. 3 depicts the fluid source 40 coupled to a fluid port 41 of the accessory medical device 14 via a fluid tube 42 and a flow regulator 44, the fluid source 40 and/or other suitable fluid sources (e.g., syringes, etc.) may be coupled to the fluid port 41 in other suitable manners. In one example, a syringe with contrast fluid may be coupled to the fluid port 41 via a luer lock connection and/or other suitable connection. Contrast agent may be delivered into the body lumen and may be used to temporarily improve imaging of the inside of the body lumen by, for example, x-ray, computed tomography (CT), or magnetic resonance (MR) imaging, ultrasound, and the like. Alternatively, separate lumens and separate fluid ports may be provided for each of a pressurized fluid and a contrast fluid.

A further lumen of the one or more lumens may be configured to accept the guidewire 12 and/or one or more other suitable medical devices usable with the accessory medical device 14. The further lumen may extend from the distal end 26b of the tube 26 (e.g., an opening at the distal tip 31) at least partially along the tube 26 toward the proximal end 26a of the tube 26.

In some cases, the guidewire 12 or other suitable medical devices may extend through the further lumen of the elongate tube 26 and proximally out of an aperture port 46 (e.g., a side port). The aperture port 46 may be located at the distal end of the handle 28 and/or at one or more other suitable locations. In some cases, the aperture port 46 and the tube 26 may be configured so that the guidewire 12 and/or other suitable medical devices may be stripped through the side of the tube 26 for a rapid removal or exchange of devices, but this is not required.

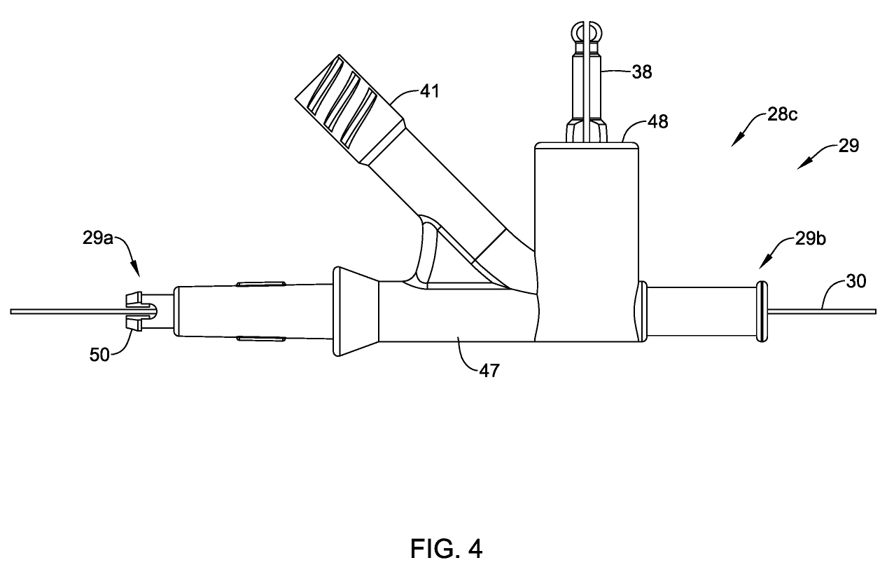

FIG. 4 schematically depicts a side view of an illustrative configuration of the third portion 28c of the handle 28 or hub 29, with the wire 30 extending therethrough. As depicted in FIG. 4, the hub 29 may include the fluid port 41 and the electrical connector 38 at an electrical connector port 48. In some examples, the electrical connector 38 and the electrical connector port 48 may be distal of the fluid port 41, but other configurations are contemplated in which the electrical connector port 48 and the electrical connector 38 are proximal of the fluid port 41. In some examples, the hub 29 may include a housing 47 defining the fluid port 41, the electrical connector port 48, and one or more lumens.

The housing 47 of the hub 29 may be formed from one or more components. In some examples, the housing 47 may be formed from a single monolith component formed from a single material. In some examples, the housing 47 may be formed from a plurality of components coupled to one another in any suitable manner including, but not limited to, a hinged connection, a snap connection, an adhesive connection, an over-molded connection, and/or other suitable type of connection.

The hub 29 may include a proximal end 29a and a distal end 29b. The distal end 29b may be configured to couple with the tube 26 (not shown in FIG. 4) and the proximal end 29a may include a connector 50 configured to couple with the actuator 32 (e.g., the first portion 28a and/or the second portion 28b of the handle 28). The hub 29 may include one or more openings at the proximal end 29a and one or more openings at the distal end 29b configured to receive the wire 30, fluid, and/or other components of and/or used with the accessory medical device 14.

The connector 50 may be any suitable type of connector 50. For example, the connector 50 may be a press-fit connector, a snap connector, a rotational connector, a threaded connector, a ball-detent connector, and/or other suitable type of connector. In one example, the connector 50 may be a snap and rotational connector, where the connector 50 may create a snap connection between the second portion of 28b of the handle 28 and the hub 29, while allowing the second portion 28b to rotate relative to the hub 29. In some examples, the connector 50 may be configured to longitudinally fix the second portion 28b of the handle 28 relative to the hub 29.

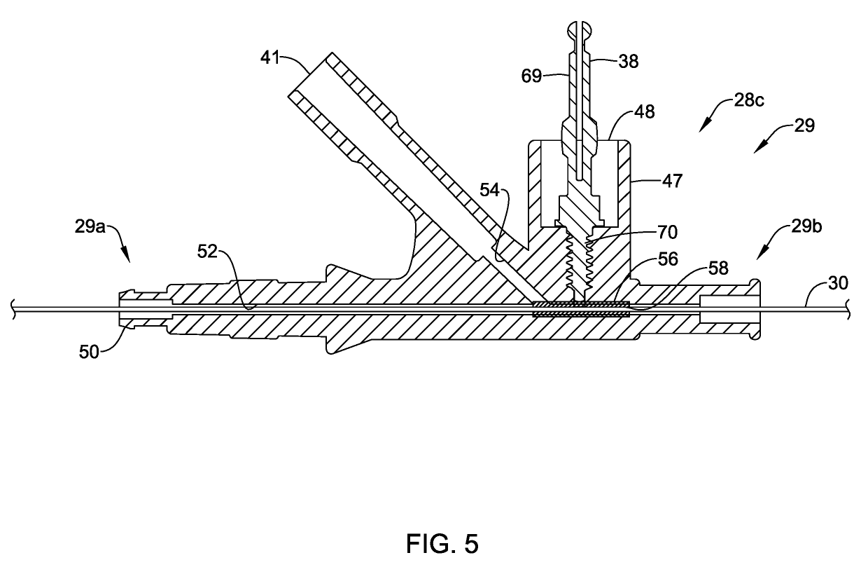

FIG. 5 depicts a schematic cross-section view of configuration of the hub 29 depicted in FIG. 4, with the wire 30 shown in a side view extending through a wire lumen 52 extending from the proximal end 29a of the hub 29 to the distal end 29b of the hub 29. In addition to the wire lumen 52, the hub 29 may include and/or define one or more other lumens including, but not limited to, a fluid lumen 54 of or in fluid communication with the fluid port 41 and extending from the fluid port 41 to the distal end 29b of the hub 29. The fluid lumen 54 may be fluidly coupled with the wire lumen 52 and/or may be entirely separate and electrically insulated from the wire lumen 52.

The electrical connector 38 may have a connector portion 69 (e.g., a first portion) configured to mechanically and/or electrically couple with a power source and/or a component in communication with a power source and a tip portion 70 (e.g., a second portion) configured to mechanically and/or electrically couple with the conductive tube 56. The electrical connector 38 may be coupled with the hub 29 at the electrical connector port 48 in any suitable manner. For example, the electrical connector 38 may be configured to couple with the hub 29 via a threaded coupling at the tip portion 70, an adhesive coupling, a fastener (e.g., nut and/or other suitable fastener) configured to engage the tip portion 70, a friction fit, a press-fit, a detent coupling, a snap coupling, and/or other suitable type of coupling. In one example, the electrical connector 38 may be coupled with the housing 47 of the hub 29 via a threaded connection utilizing threads along the tip portion 70 of the electrical connector 38, as depicted for example in FIG. 5.

An electrically conductive tube 56 having a tube lumen 58 may be located within the hub 29 along the wire lumen 52. In some examples, the tube lumen 58 may be axially aligned with a central axis of the wire lumen 52. The conductive tube 56 may be positioned along the hub 29 at a location that is in electrical communication or contact with the electrical connector 38 when the electrical connector 38 is coupled with the hub 29. In some examples, a distal end of the electrical connector 38 may be electrically coupled with the conductive tube 56 (e.g., coupled with an outer surface of the conductive tube56), as depicted for example in FIG. 5.

The tube lumen 58 may have any suitable configuration configured to receive the wire 30 and transfer electrical power from the electrical connector 38 to the wire 30, while allowing the wire 30 to longitudinally translate and/or rotate relative to the conductive tube 56. For example, the wire 30 may be slidingly arranged with respect to the tube lumen 58 so as to electrically couple with the conductive tube 56 and the electrical connector 38. Although the conductive tube 56 may have a constant inner surface defining an inner diameter and the tube lumen 58, the conductive tube 56 may include one or more protrusions, bends, and/or components at one or more longitudinal and/or circumferential locations that are configured to electrically couple with the wire 30 as the wire longitudinally and/or rotationally translates with respect to the conductive tube 56 in response to adjustment of the first and/or second portions 28a, 28b of the handle 28 (not shown in FIG. 5) relative to the hub 29. In some examples, one or more electrically conductive components may be fixedly coupled with the wire 30 and configured to electrically couple the conductive tube 56 and the wire 30.

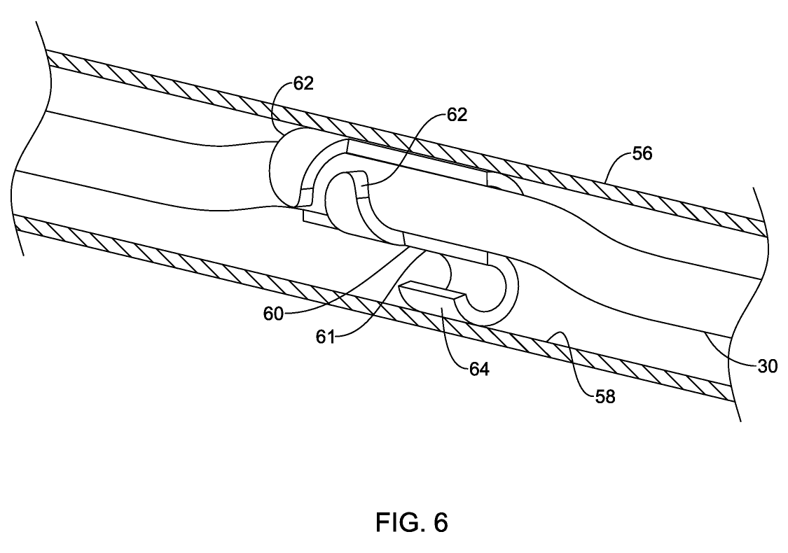

FIG. 6 depicts a schematic view of the conductive tube 56, the wire 30, and an illustrative configuration of a crimp connector 60, with the conductive tube 56 depicted in cross-section and the wire 30 and the crimp connector 60 depicted from a side view. The crimp connector 60 may be configured to be coupled with the wire 30 and electrically engage an inner surface of the conductive tube 56. In some examples, the crimp connector 60 may be fixedly secured to the wire 30 such that the crimp connector 60 adjusts or translates with the wire 30 within the conductive tube 56.

The crimp connector 60 may have any suitable configuration for coupling with the wire 30, engaging the inner surface of the conductive tube 56, and/or otherwise electrically coupling the electrical connector 38 with the wire 30. In some examples, the crimp connector 60 may include a body 61, one or more fixation components 62, and one or more extension components 64 (e.g., whiskers, brushes, flexible sheets, etc.) configured to be in continuous contact with the inner surface of the conductive tube 56 during operation of the accessory medical device 14. The one or more fixation components 62 may extend from the body 61 and may be secured to the wire 30. The one or more extension components 64 may extend from the wire 30 and/or the body 61 to the inner surface of the conductive tube 56. In one example and as depicted for example in FIG. 6, the crimp connector 60 may include two fixation components longitudinally and circumferentially spaced from one another and secured around the wire 30 and one extension component 64 longitudinally and circumferentially spaced from the fixation components 62. Other suitable configurations of the crimp connector 60 are contemplated. As the body 61 of the crimp connector 60 may be offset from a central axis of the conductive tube 56, the wire 30 may include one or more bends proximal of and proximate to the crimp connector 60 and/or distal of and proximate to the crimp connector 60, as depicted for example in FIG. 6, such that the wire 30 may be centrally positioned within the tube lumen 58 of the conductive tube 56 to facilitate longitudinal and/or rotational translation or adjustment of the wire 30 relative to the conductive tube 56.

The one or more extension components 64 may extend from one or more sides of the body 61. In some examples, the extension components 64 may be configured about the one or more sides of the body 61 to offset the body 61 and/or the wire 30 from a central axis of the tube lumen 58 of the conductive tube 56. In some examples, the one or more extension components 64 may be configured about the one or more sides of the body 61 to center the body 61 and/or the wire 30 at a central axis of the tube lumen 58 of the conductive tube 56.

The one or more extension components 64 may have any suitable configuration. For example, the one or more extension components 64 may have a curved shape for engaging a curved-shape of an inner surface of the conductive tube 56, bristles, flexible sheets, a C-shape, an S-shape, and/or other suitable shape. In some examples, one or more of the extension components 64 may have a curved C-shape configured to engage the inner surface of the conductive tube 56 such that the extension components 64 are elastically deformed and applying an outward radial force on the inner surface of the conductive tube 56. Other suitable configurations of the extension components 64 are contemplated.

The crimp connector 60 may be secured to the wire 30 in any suitable manner. For example, the crimp connector 60 may be secured to the wire 30 via a crimp connection, a solder connection, and/or other suitable mechanical and/or electrical connections. In some examples, the one or more fixation components 62 of the crimp connector 60 may extend from the body 61, may extend at least partially circumferentially around the wire 30, and may be secured to the wire 30 via a crimp connection, a weld connection, and/or a solder connection.

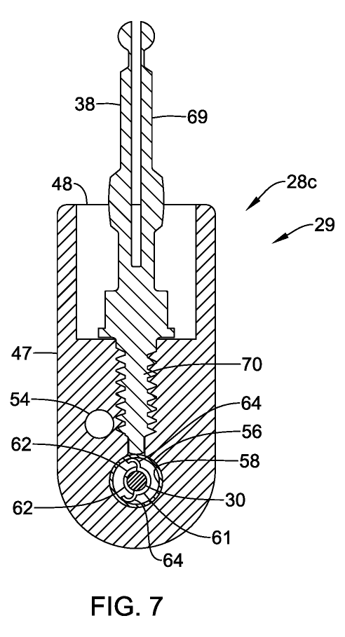

FIG. 7 depicts a schematic view of an illustrative configuration of the hub 29, with the crimp connector 60 shown from an end view and the housing 47, the wire 30, the conductive tube 56, and the electrical connector 38 shown in cross-section as the crimp connector 60 is offset in a distal direction from the electrical connector 38. The illustrative configuration of the crimp connector 60 depicted in FIG. 7 may include two fixation components 62 extending from the body 61 and coupled with the wire 30 and two extension components 64 extending from the body 61 and electrically coupled with the inner surface of the conductive tube 56. When the crimp connector 60 includes two or more extension components 64, the extension components 64 may be sized such that the wire 30 is centrally located within the tube lumen 58 of the conductive tube 56, as depicted for example in FIG. 7, and able to longitudinally and/or rotationally adjust within the tube lumen 58 with the crimp connector 60 as the extension components 64 apply a force to the inner surface of the conductive tube 56. Other suitable configurations of the crimp connector 60 are contemplated.

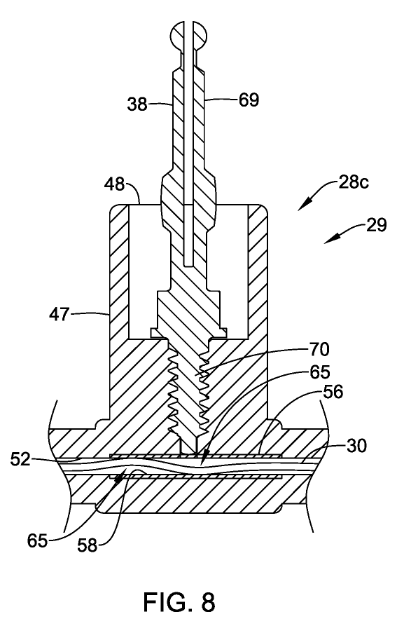

FIG. 8 depicts a schematic view of an illustrative configuration of a portion of the hub 29, with the wire 30 shown from a side view and the housing 47, the wire 30, and the electrical connector 38 shown in cross-section. As depicted in FIG. 8, the crimp connector 60 may be omitted and the wire 30 may be configured to contact the inner surface of the conductive tube 56 and maintain an electrical connection with the electrical connector 38 via the conductive tube 56 during operation of the accessory medical device 14.

The wire 30 may have any suitable configuration configured to contact the inner surface of the conductive tube 56 while permitting longitudinal and/or rotational translation of the wire 30 relative to the conductive tube 56. For example, at a location of the wire 30 configured to remain within the tube lumen 58, the wire 30 may have one or more bends 65, curved shapes in the longitudinal direction of the wire 30, an S-shape, an outer diameter configured to contact the inner surface of the conductive tube 56 that is larger than outer diameter configured to remain exterior of the conductive tube 56, and/or other suitable configurations configured to contact an inner surface of the conductive tube 56. In one example, at a location of the wire 30 configured to remain within the tube lumen 58, the wire 30 may have one or more bends 65, in the form of an S-curve, configured to contact the inner surface of the conductive tube 56, as depicted in FIG. 8.

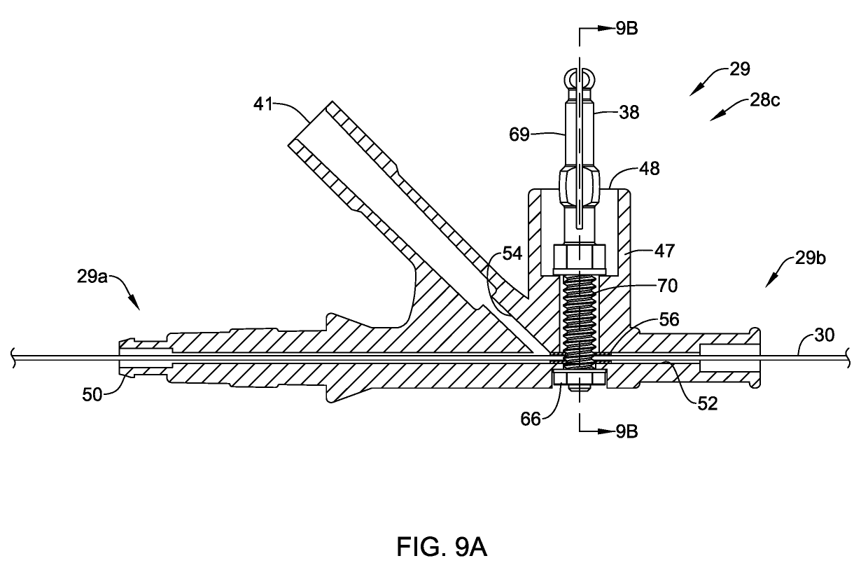

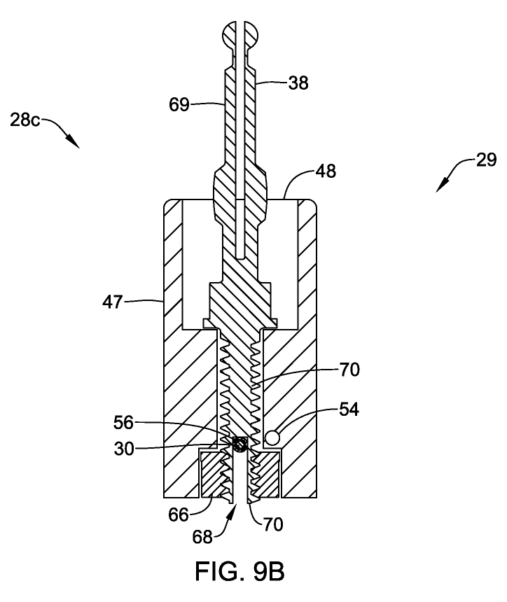

FIGS. 9A-16 depict illustrative configurations of the electrical connector 38 and the conductive tube 56 within the hub 29 to electrically couple the electrical connector 38 and the wire 30 via the conductive tube 56 and electrically insulate the fluid lumen 54 from the electrical connector 38, the conductive tube 56, and the wire 30. Turning first to FIGS. 9A and 9B, FIGS. 9A and 9B depict schematic partial cross-section views of an illustrative configuration of the hub 29 in which a fastener 66 (e.g., a nut, etc.) or other suitable locking component may be coupled with the electrical connector 38 such that the conductive tube 56 is secured therebetween. As depicted in FIG. 9A, the housing 47 of the hub 29 and the conductive tube 56 are schematically depicted in a longitudinal cross-section and the electrical connector 38, the wire 30, and the fastener 66 are depicted from a schematic side view. All components in FIG. 9B are schematically depicted in cross-section taken at an axial location of the electrical connector 38 (e.g., line 9B-9B in FIG. 9A).

In the configuration of the hub 29 depicted in FIGS. 9A and 9B, the electrical connector 38 may extend over the conductive tube 56 through which the wire 30 extends. The fastener 66 may be threadedly coupled with external and/or internal threads of the electrical connector 38 such that the conductive tube 56 may be secured within the hub 29 between the electrical connector 38 and the fastener 66. In some examples, coupling the fastener 66 with the electrical connector 38 when the conductive tube 56 is therebetween, may fix the electrical connector 38 relative to the housing 47 and/or other components of the hub 29.

As depicted in FIG. 9B, the tip portion 70 of the electrical connector 38 may extend into a hole (e.g., a through-hole and/or other suitable hole) of the housing 47 and of or in communication with the electrical connector port 48 of the hub 29 and include a slot 68 extending through a distal end of the electrical connector 38, where the slot 68 may be configured to receive the conductive tube 56. Once the conductive tube 56 has been received within the slot 68, the fastener 66 and/or other suitable locking component may be engaged with (e.g., via threads, etc.) the electrical connector 38 via the hole in the hub 29 to secure the conductive tube 56 in electrical communication with the electrical connector 38 and secure the electrical connector 38 at and/or within the housing 47 of the hub 29. In some examples, the fluid lumen 54 may be electrically insulated (e.g., by the housing 47) from and extend along the conductive tube 56 and/or the wire lumen 52, as depicted for example in FIG. 9B, but other suitable configurations are contemplated.

The configuration of the hub 29 depicted in FIGS. 9A and 9B may be assembled in any suitable manner. In some examples, the housing 27 may be a pre-formed single component or monolithic component formed from a single material and the conductive tube 56 may be inserted into the housing 47 through the wire lumen 52 at the proximal end 29a and/or the distal end 29b of the hub 29 to a location proximate the electrical connector 38 for electrically coupling the conductive tube 56 with the electrical connector 38. The electrical connector 38 may be inserted into the electrical connector port 48 and slide into contact with and/or over the conductive tube 56 such that the conductive tube 56 may be received within the slot 68 and electrically coupled with the electrical connector 38. In some examples, the conductive tube 56 may be pinched within the slot 68. Alternatively or additionally, the electrical connector 38 may be inserted into the electrical connector port 48 prior to inserting the conductive tube 56 into the housing 47 such that the conductive tube 56 is inserted into the slot 68 as it is positioned within the housing 47. Once the conductive tube 56 is located within the slot 68, the fastener 66 and/or other suitable locking component may be engaged with the electrical connector 38 and the conductive tube 56 to ensure a mechanical and electrical connection between the electrical connector 38 and the conductive tube 56. To prevent the fastener 66 and/or other locking component from backing out, the fastener 66 and/or other locking component may be covered with a plug, a cured adhesive, and/or other suitable securing component. The wire 30 may be inserted into the conductive tube 56 at any suitable time before, during, and/or after assembling the hub 29. Other suitable techniques for assembling the configuration of the hub 29 depicted in FIGS. 9A and 9B are contemplated.

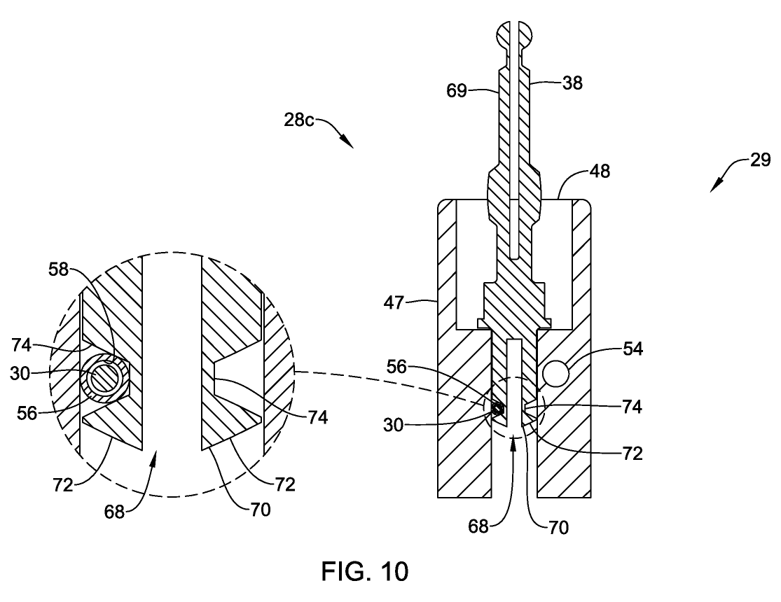

FIG. 10 depicts a schematic cross-section view of an illustrative configuration of the hub 29 in which the electrical connector 38 is fixed within the hole in the housing 47 of the hub 29 via a flex-fit, press-fit, and/or snap connection with the conductive tube 56 and an inner surface of the hole in the housing 47. For example, the conductive tube 56 may be inserted into the housing 47 (e.g., via the wire lumen 52) before or after inserting the electrical connector 38 and the electrical connector 38 may engage the conductive tube 56 and the inner surface of the hole of the housing 47 to fix (e.g., removably fix or non-removably fix) the electrical connector 38 within the housing 47 of the hub 29.

The electrical connector 38 may include one or more slots 68 in the tip portion 70, where the slots 68 and/or the material of the electrical connector 38 may define one or more legs and facilitate flexing of the tip portion 70. The legs of the electrical connector 38 at the tip portion 70 may include one or more barbs or protrusions 72 and/or recesses 74 that may engage and/or receive the conductive tube 56 to electrically couple the conductive tube 56 and the wire 30 with the electrical connector 38 and mechanically couple the electrical connector 38 with the conductive tube 56 via a press-fit, as depicted for example in FIG. 10. The engagement of the barb or protrusion 72 with the conductive tube 56 may prevent or mitigate unintentional removal of the electrical connector 38 relative to the housing 47 and/or the conductive tube 56.

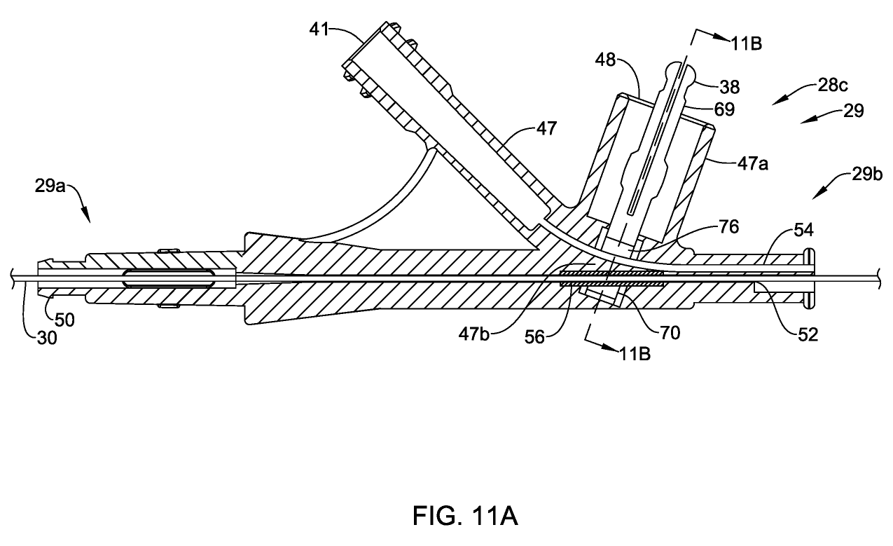

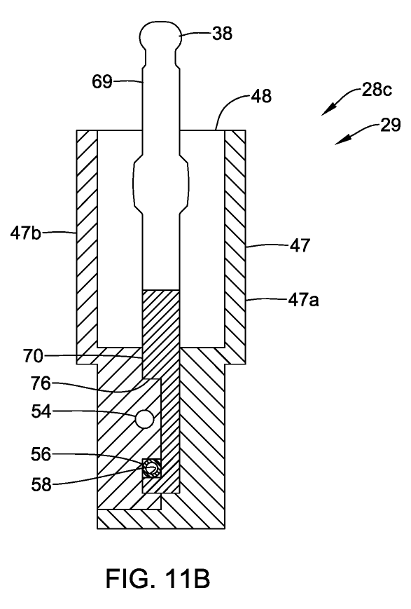

FIGS. 11A and 11B depict schematic partial cross-section views of an illustrative configuration of the hub 29 in which the tip portion 70 of the electrical connector 38 may include a recess 76 for receiving the conductive tube 56 and a portion of the housing 47 defining or through which the fluid lumen 54 extends. FIG. 11A schematically depicts the components of the hub 29 at a first portion 47a of the housing 47 in a longitudinal cross-section view and FIG. 11B schematically depicts the components of the hub 29 at the first portion 47a and a second portion 47b in a cross-section view taken along line 11B-11B in FIG. 11A, but with the fluid lumen 54 in the second portion 47b rather than in the first portion 47a of the housing 47.

In the configuration of the hub 29 depicted in FIGS. 11A and 11B, the recess 76 may be configured to receive the second portion 47b of the housing 47 defining and/or including the fluid lumen 54 and receive the conductive tube 56, such that the housing 47 defining and/or including the fluid lumen 54 may be electrically insulated from the conductive tube 56 and/or the electrical connector 38. The fluid lumen 54 may be electrically insulated form the conductive tube 56 via the housing 47, an insulated tube extending around the fluid lumen 54, and/or electrically isolated from the conductive tube 56 in any suitable manner. The conductive tube 56 may be secured within the recess 76 between the housing 47 and the electrical connector 38 so as to be in electrical communication with the electrical connector 38.

The electrical connector 38 and/or the conductive tube 56 may be secured in the housing 47 in any suitable manner. For example, the electrical connector 38 and/or the conductive tube 56 may be secured within the housing 47 by over-molding the housing 47 onto the tip portion 70 of the electrical connector 38 and the conductive tube 56, using a two or more-piece housing 47 where the pieces of the housing 47 are connectable to one another in a secure manner, and/or secured within the housing 47 in one or more other suitable manners. In one example and as depicted in FIG. 11B, the housing 47 may have the first portion 47a and the second portion 47b, which may be press-fit together, snapped together, coupled via an adhesive, coupled via a clam shell connection, and/or coupled in one or more other suitable manners. As depicted in FIG. 11B, the second portion 47b of the housing 47 may define the fluid lumen 54 and may be inserted into the recess 76 of the tip portion 70 of the electrical connector 38 and create a pressure fit mechanical connection with the conductive tube 56 and the electrical connector 38 such that the conductive tube 56 and the electrical connector 38 are in electrical communication with one another. Although the fluid lumen 54 is depicted in FIG. 11B as being formed in the second portion 47b of the housing 47 at a location that is inserted within the recess 76 of the electrical connector 38, the fluid lumen 54 may be formed in and/or otherwise located in the first portion 47a (e.g., as depicted in FIG. 11A) or the second portion 47b at any suitable location along the housing 47 and in fluid communication with the fluid port 41.

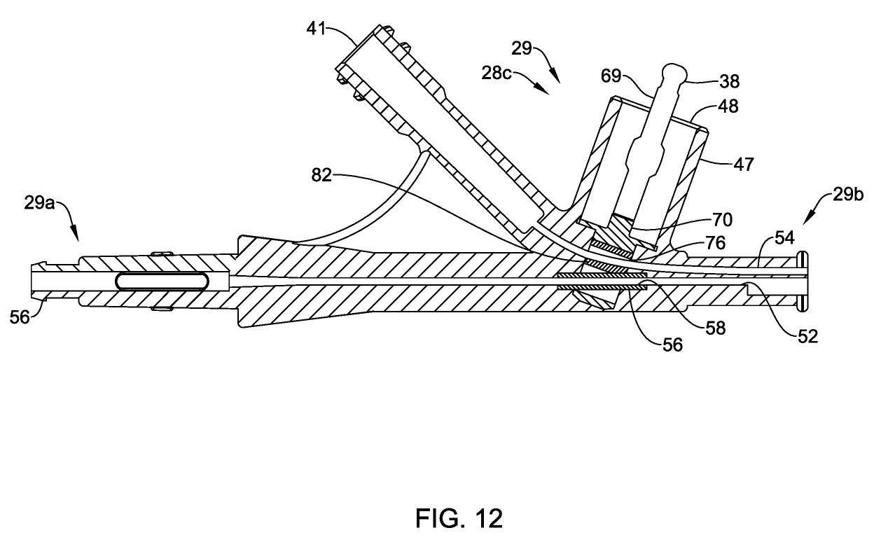

FIG. 12 depicts a schematic cross-section view of an illustrative configuration of the hub 29 in which the electrical connector 38 may be formed with the recess 76 in the tip portion 70, where the recess 76 may be configured to receive the fluid lumen 54 and the conductive tube 56. In some examples, the recess 76 of the electrical connector 38 may be configured to receive an insulating sleeve or tube 82 configured to define the fluid lumen 54 and/or otherwise receive the fluid lumen 54 (e.g., via a fluid lumen tube, etc.). The insulating tube 82 may be configured such that when the insulating tube 82 is within the recess 76, the insulating tube 82 engages an outer surface of the conductive tube 56 and press-fits or otherwise urges the conductive tube 56 against the electrical connector 38 to create and/or maintain an electrical connection between the electrical connector 38 and the conductive tube 56.

The configuration of the hub 29 depicted in FIG. 12 may be assembled in any suitable manner. In some examples, the insulating tube 82 and the conductive tube 56 may be wedged into the recess 76 of the electrical connector 38 prior to coupling the electrical connector 38 and/or the conductive tube 56 with the housing 47. Alternatively or additionally, one or more of the electrical connector 38, the conductive tube 56, and the insulating tube 82 may be coupled with the housing 47 prior to coupling with one or more other of the electrical connector 38, the conductive tube 56, and the insulating tube 82. The housing 47 may be coupled with the electrical connector 38, the conductive tube 56, and the insulating tube 82 in any suitable manner discussed herein or otherwise.

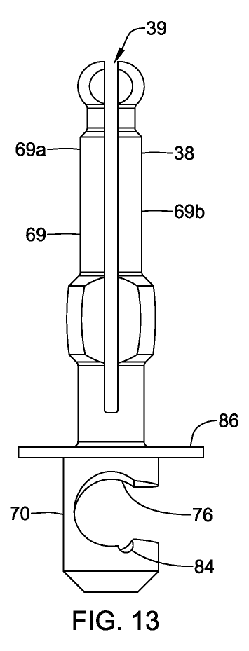

FIG. 13 schematically depicts a side view of an illustrative configuration of an electrical connector 38 usable with the configuration of the hub 29 depicted in FIG. 12 and/or other suitable configurations of the hub 29. As depicted in FIG. 13, the electrical connector 38 may include a divider or plate 86 between the connector portion 69 and the tip portion 70. The plate 86 may be configured to limit insertion of the electrical connector 38 within the electrical connector port 48, may facilitate positioning the electrical connector 38 in a mold when the housing 47 is over-molded around the electrical connector 38, may facilitate removing the electrical connector 38 from a mold after forming the electrical connector 38, and/or may be used for one or more other suitable purposes.

The connector portion 69 of the electrical connector 38 may have any suitable configuration for coupling with a power source or component in communication with the power source. In one example configuration, the connector portion 69 may have a plurality of prongs or arms 69a, 69b separated by one or more slots 39. When included, the prongs or arms 69a, 69b may include a catch 71 for receiving and coupling with the power source or component in communication with the power source. Other suitable configurations of the connector portion 69, as disclosed herewith or otherwise, are contemplated.

The tip portion 70 of the electrical connector 38 may include the recess 76 configured to receive the conductive tube 56 and the insulating tube 82 (e.g., as depicted in FIG. 12). As depicted in FIG. 13, the electrical connector 38 may include a secondary opening 84 extending from or part of the recess 76. The secondary opening 84 may be configured to receive the conductive tube 56 via the recess 76. The secondary opening 84 may be positioned and/or configured relative to the recess 76 such that when the conductive tube 56 is within the secondary opening 84 and the insulating tube 82 is within the recess 76, the insulating tube 82 may apply a force to the conductive tube 56 to maintain electrical communication between (e.g., maintain an electrically conductive contact between) the conductive tube 56 and the electrical connector 38. Other suitable configurations of the tip portion 70, as disclosed herewith or otherwise, are contemplated.

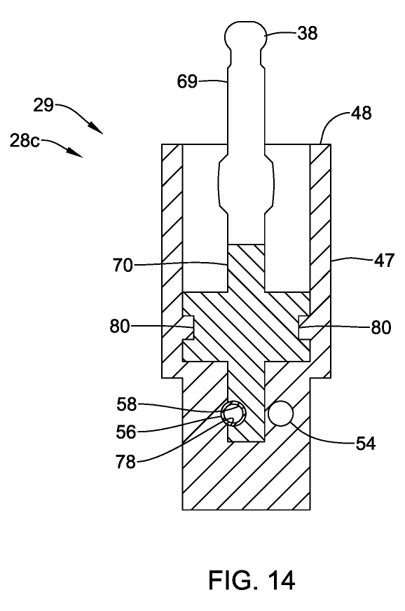

FIG. 14 depicts a schematic cross-section view of an illustrative configuration of the hub 29 in which the electrical connector 38 may be formed with an opening 78 configured to receive the conductive tube 56. The opening 78 may define a full circumference of a circle or a partial circle or other shape configured to receive and mate with the conductive tube 56. In some examples, the opening 78 may be more than a half of a circumference of an enclosed shape, such as a circle, so as to create a snap or other suitable press-fit connection with the conductive tube 56.

The electrical connector 38 may include one or more fixation components 80. In some examples, the fixation components 80 may be recesses, protrusions, and/or other suitable fixation components. The fixation components 80 may extend entirely around a circumference of the electrical connector 38 and/or partially around the electrical connector 38. In one example and as depicted in FIG. 12, the electrical connector 38 may include a fixation component 80 configured as a recess extending entirely around a circumference of the electrical connector 38 and may receive material of the housing 47 to mechanically secure the electrical connector 38 within the housing 47. The housing 47 may be formed in any suitable manner discussed herein or otherwise and the electrical connector 38 and the conductive tube 56 may be coupled with the housing 47 in any suitable manner discussed herein or otherwise.

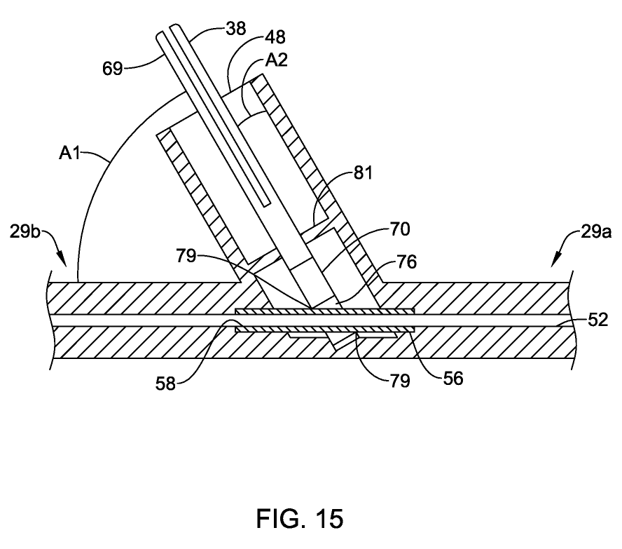

FIG. 15 depicts a schematic partial cross-section view of an illustrative configuration of the hub 29 in which the housing 47 and the conductive tube 56 are depicted in cross-section and the electrical connector 38 is depicted from a side view. The electrical connector 38 may be formed with the recess 76 configured to receive the conductive tube 56 and may be coupled with the housing 47 of the hub 29.

The recess 76 and the coupling between the electrical connector 38 and the housing 47 may be configured such that the portion of the electrical connector 38 defining the recess 76 may apply a pinch-force to or otherwise have contact points 79 at one or more locations with the conductive tube 56. In one example configuration of the hub 29, the recess 76 may be partially defined by top and bottom limits that are perpendicular to a longitudinal axis of the electrical connector 38 (e.g., the recess may be perpendicular to the longitudinal axis of the electrical connector 38) and the electrical connector 38 may be configured to couple with the housing 47 so as to be at a non-perpendicular angle A1with a longitudinal axis extending through the wire lumen 52. Alternatively or additionally, the electrical connector port 48 may be perpendicular to or at one or more other suitable angles with respect to the longitudinal axis extending through the wire lumen 52 and the electrical connector 38 may be coupled with the housing 47 via the electrical connector port 48 such that the electrical connector 38 is at an angle A2 with respect to the electrical connector port 48. Alternatively or additionally and although not shown in FIG. 15, the top and/or bottom limits of the recess 76 may be configured to be at a non-parallel angle with respect to the conductive tube 56 or at a non-perpendicular angle with respect to a longitudinal axis extending through the electrical connector 38. When the recess 76 and the electrical connector 38 are so configured and/or arranged, the electrical connector 38 may apply a pinch-force to or have contact points 79 with the conductive tube 56 when the conductive tube 56 is positioned within the recess 76, as depicted for example in FIG. 15.

The housing 47 may include one or more supports 81 configured to engage the electrical connector 38. For example, the supports 81 may be located at the fluid port 48 and configured to engage the electrical connector 38 at a location proximal of the recess 76 to facilitate maintaining the electrical connector 38 at the desired angle A1 and/or angle A2. The supports 81 may take on any suitable configuration configured to support the electrical connector 38 at the desired angle relative to the longitudinal axis of the wire lumen 52 and/or the tube lumen 58.

The angle A1 and/or the angle A2 may be any suitable angle. In some examples, the angle A1 and/or the angle A2 may be any suitable angle less than 90 degrees. In one example, the angle A1 may be 85 degrees and/or the angle A2 may be 5 degrees and/or any other suitable angle.

The configuration of the hub 29 depicted in FIG. 15 may be assembled in any suitable manner. In some examples, when over-molding the housing 47 onto the electrical connector 38 and the conductive tube 56, the electrical connector 38 may be slid over the conductive tube 56, the electrical connector 38 may then be angled with respect to the conductive tube 56 to create the pinch points or contact points 79, and then the housing 47 may be over-molded onto the electrical connector 38 and the conductive tube 56. In some examples, when housing 47 is pre-formed (e.g., pre-molded into one or more pieces), the electrical connector 38 may be inserted into a desired position within the electrical connector port 48 and the conductive tube 56 may be press-fit into the recess 76 of the tip portion 70 of the electrical connector 38 to create the pinch points or contact points 79 and an electrical coupling between the electrical connector 38 and the conductive tube 56. Other suitable configurations for assembling the configuration of the hub depicted in FIG. 15 are contemplated.

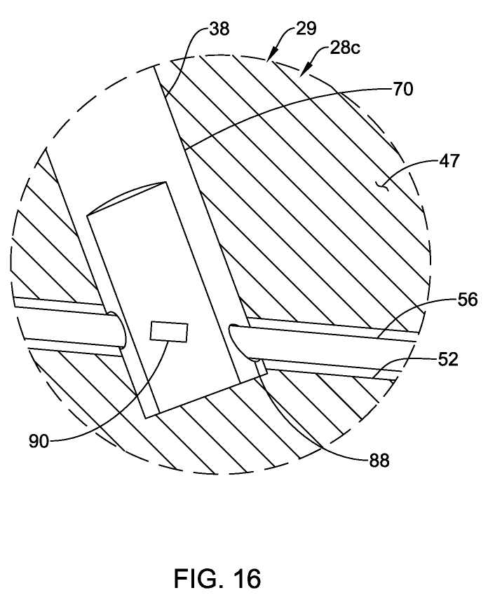

FIG. 16 depicts a schematic partial cross-section view of a portion of an illustrative configuration of the hub 29 in which the housing 47 is depicted in cross-section, while the electrical connector 38 and the conductive tube 56 are depicted in a side view. The conductive tube 56 extending through and/or in communication with the wire lumen 52 may be inserted through an opening 88 (e.g., a through-hole and/or other suitable opening) in the tip portion 70 of the electrical connector 38. In some examples, the opening 88 may be at a perpendicular or non-perpendicular angle with respect to a longitudinal axis of the electrical connector 38. To mechanically and/or electrically couple the conductive tube 56 with the electrical connector 38, one or more crimps 90 may be created in the electrical connector 38 at a location proximate the conductive tube 56. In some examples, the one or more crimps 90 may be configured to engage, but not deform the conductive tube 56.

The configuration of the hub 29 depicted in FIG. 16 may be assembled in any suitable manner. In some examples, when over-molding the housing 47 onto the electrical connector 38 and the conductive tube 56, the electrical connector 38 may be slid over the conductive tube 56 such that the conductive tube 56 is received within the opening 88 of the electrical connector 38, the one or more crimps 90 may then be applied to the electrical connector 38 to mechanically and/or electrically couple the electrical connector 38 with the conductive tube 56, and then the housing 47 may be over-molded onto the electrical connector 38 and the conductive tube 56. In some examples, when housing 47 is pre-formed (e.g., pre-molded into one or more pieces), the electrical connector 38 may be inserted into a desired position within the electrical connector port 48 and the conductive tube 56 may be slide into and/or press-fit into the opening 88 via the wire lumen 52. The pre-formed housing 47 may include one or more holes aligned with one or more locations along the electrical connector 38 and the conductive tube 56 at which the one or more crimps 90 are to be formed in the electrical connector 38. As such, when the electrical connector 38 and the conductive tube 56 are positioned at a desired location within the housing 47, the one or more crimps 90 may be applied to the electrical connector 38 through the holes in the preformed housing 47. After mechanically and/or electrically coupling the electrical connector 38 and the conductive tube 56 via the one or more crimps 90, the holes may or may not be filled with a material of the housing, a resin, electrically insulative material, and/or other suitable material. Other suitable configurations for assembling the configuration of the hub 29 depicted in FIG. 16 are contemplated.

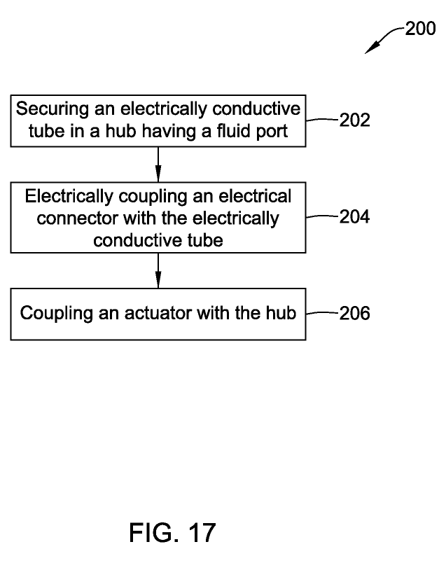

Any suitable methods for assembling the accessory medical device 14 may be utilized, which includes the methods discussed herein with respect to the various configurations of the hub 29 and the accessory medical device 14 and/or other suitable methods. FIG. 17 depicts an illustrative configuration of a method 200 for assembling a medical device (e.g., the accessory medical device 14 and/or other suitable medical devices).

The method 200 may include securing 202 an electrically conductive tube in a hub of a medical device. In some examples, the hub may include a fluid port. When the hub is included within a handle of the medical device, the hub may be configured to be stationary relative to one or more control portions of the handle that are configured to be rotationally and/or longitudinally translated relative to the hub.

The electrically conductive tube may be secured in the hub in any suitable manner, as discussed herein or otherwise. For example, the electrically conductive tube made be secured in the hub using one or more techniques including, but not limited to, over-molding the electrically conductive tube in the hub, press-fitting the electrically conductive tube in the hub, coupling two or more components of the hub around the electrically conductive tube, pinching the conductive tube between the housing and the electrical connector, and/or engaging the electrically conductive tube between the electrical connector and a fastener coupled with the electrical connector.

The method 200 may include electrically coupling 204 electrically conductive tube with the electrical connector. Any suitable technique may be utilized for electrically coupling the electrically conductive tube and the electrical connector with one another, as discussed herein or other otherwise. For example, the electrically conductive tube may be electrically coupled with the electrical connector using one or more techniques including, but not limited to, engaging the electrically conductive tube between the electrical connector and a fastener coupled with the electrical connector, crimping the electrical connector onto the conductive tube, clamping or positioning the conductive tube between the electrical connector and the housing the hub, pinching the conductive tube within a recess of the electrical connector, and/or press-fitting the conductive tube into a recess or opening of the electrical connector.

The method 200 may include coupling 206 an actuator with the hub. The actuator may be configured to longitudinally, rotationally, and/or otherwise adjust or translate relative to the hub when coupled with the hub. The actuator may be coupled with the hub in any suitable manner including, but not limited to, using a press-fit connection, a snap connection, a rotational connection, a threaded connection, a detent connection, and/or other suitable type of connection.

The actuator may be coupled with a conductive wire and/or suitable conductive component configured to be in electrical communication with a power source via the electrical connector and/or the conductive tube. In one example in which the accessory medical device may be a sphincterotome, the conductive component may be a wire extending from the actuator to a distal end of a tube coupled with a distal end of the hub. The wire may extend through the conductive tube and may be electrically coupled with the tube, as discussed herein or otherwise, and may longitudinally and/or rotationally translate relative to the conductive tube in response to actuation of the actuator. The actuator coupled with the hub and the wire may facilitate forming a handle of the medical device.

Other suitable components may be coupled to the hub to form the medical device. For example, an elongate tube may be coupled with the hub, one or more control wires may be coupled with the hub, and/or other suitable components may be coupled with the hub.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure’s scope is, of course, defined in the language in which the appended claims are expressed.

What is claimed is:

1. A handle for a medical device configured to connect with an electrical source, the handle comprising:
a hub, the hub comprising:
a fluid port;
an electrical connector; and
an electrically conductive tube coupled with the electrical connector;
a wire electrically coupled to the electrically conductive tube; and
an actuator coupled with the hub and the wire, wherein the actuator is configured to adjust relative to the hub and in response, adjust the wire relative to the electrically conductive tube.

2. The handle of claim 1, wherein the wire comprises a bend configured to engage an inner surface of the electrically conductive tube.

3. The handle of claim 1, further comprising:
a crimp connector coupled with the wire, wherein the crimp connector is configured to engage an inner surface of the electrically conductive tube.

4. The handle of claim 1, wherein the electrical connector is located distal of the fluid port and a lumen in communication with the fluid port is electrically isolated from the electrical connector.

5. The handle of claim 1, wherein the electrical connector comprises threads configured to engage the hub and a distal end of the electrical connector is electrically coupled with the electrically conductive tube.

6. The handle of claim 1, wherein the wire is configured to longitudinally and rotationally adjust relative to the electrically conductive tube.

7. The handle of claim 6, wherein:
the electrical connector comprises a slot extending through a distal end of electrical connector;
the hub comprises a through-hole configured to receive the electrical connector;
the slot is configured to receive the electrically conductive tube; and
a fastener is configured to be positioned within the through-hole and engage threads extending around the electrical connector to maintain electrical contact between the electrical connector and the electrically conductive tube received within the slot.

8. The handle of claim 6, wherein:
the electrical connector comprises a slot extending through a distal end of electrical connector;
the hub comprises a hole;
a portion of the electrical connector along the slot defines two legs each having a barbed portion; and
the two legs are configured to create a press fit engagement with an inner surface of the hole and secure the electrically conductive tube at the barbed portion between the electrical connector and the inner surface of the hole.

9. The handle of claim 1, wherein the electrical connector comprises a recess configured to receive the electrically conductive tube and a portion of a housing of the hub.

10. The handle of claim 1, wherein:
the electrical connector comprises a recess configured to receive the electrically conductive tube, the recess is perpendicular to a longitudinal axis of the electrical connector;
the hub comprises an electrical connector port configured to secure the electrical connector at a non-perpendicular orientation relative to a longitudinal axis of a lumen in the hub configured to receive the wire; and
the electrical connector is configured to receive the electrically conductive tube in the recess and electrically engage an outer surface of the electrically conductive tube.

11. The handle of claim 1, wherein the electrical connector includes a crimp electrically coupling the electrical connector with the electrically conductive tube.

12. The handle of claim 1, wherein the electrical connector comprises an opening configured to receive the electrically conductive tube and electrically couple the electrical connector with the electrically conductive tube via a press-fit of the electrical connector into the opening.

13. The handle of claim 1, wherein the electrical connector is configured to receive the electrically conductive tube, a lumen in fluid communication with the fluid port, and an insulating tube positioned around the lumen and configured to urge the electrically conductive tube into electrical contact with the electrical connector.

14. A medical device comprising:
a hub having a proximal end and a distal end, the hub comprising:
a fluid port; and
an electrical connector;
an elongate shaft extending in a distal direction from the hub;
an end effector in electrical communication with the electrical connector and configured to be positioned proximate a distal end of the elongate shaft; and
an actuator coupled with the proximal end of the hub, the actuator is configured to adjust relative to the hub and in response, adjust the end effector, and
wherein the actuator and the end effector are configured to longitudinally and rotationally adjust relative to the hub.

15. The medical device of claim 14, further comprising:
a wire electrically connected with the electrical connector and mechanically connected to the actuator, and
wherein actuation of the actuator relative to the hub causes the wire and the end effector to adjust.

16. The medical device of claim 15, wherein:
the hub further comprises an electrically conductive tube electrically coupled with the electrical connector, and
the wire is electrically coupled with the electrically conductive tube and the actuation of the actuator adjusts the wire relative to the electrically conductive tube.

17. The medical device of claim 15, where the wire forms at least a portion of the end effector and is configured to cut tissue of a patient.

18. The medical device of claim 14, wherein the hub further comprises:
an electrically conductive tube electrically coupled with the electrical connector, and
a fluid lumen in communication with the fluid port and electrically insulated from the electrically conductive tube.

19. A method of assembling a medical device, the method comprising:
securing an electrically conductive tube in a hub having a fluid port;
electrically coupling an electrical connector with the electrically conductive tube; and
coupling an actuator with the hub, and
wherein the actuator coupled with the hub is configured to adjust relative to the hub.

20. The method of claim 19, wherein securing the electrically conductive tube in the hub comprises one or more of over-molding the electrically conductive tube in the hub, press-fitting the electrically conductive tube in the hub, coupling two or more components of the hub around the electrically conductive tube, and engaging the electrically conductive tube between the electrical connector and a fastener coupled with the electrical connector.
